(19) United States
(12) Patent Application Publication
(10) Pub. No.: US 2014/0336457 A1
(43) Pub. Date: Nov. 13, 2014

(54) ENDOSCOPE

(71) Applicant: Scholly Fiberoptic GmbH, Denzlingen (DE)

(72) Inventors: Matthias Kuhn, Freiburg i. Br. (DE); Jurgen Bierer, Reute (DE); Gregor Muller, Freiburg-Waltershofen (DE); Thomas Weinmann, Freiamt (DE)

(73) Assignee: SCHOLLY FIBEROPTIC GMBH, Denzlingen (DE)

(21) Appl. No.: 14/275,035

(22) Filed: May 12, 2014

(30) Foreign Application Priority Data
May 13, 2013 (DE) 202013004379.2

(51) Int. Cl. A61B 1/04; A61B 1/00

(52) U.S. Cl. CPC A61B 1/042; A61B 1/00137
USPC 600/109

(57) ABSTRACT

In the case of an endoscope 1, it is provided to arrange in an endoscope tip 3 at a distal end 2 of the endoscope 1 an imaging optic 4 and an image receiving sensor 5 in an interior space 8 defined by a metal foil 7.

### INCORPORATION BY REFERENCE

The following documents are incorporated herein by reference as if fully set forth: German Patent Application No. 202013004379.2, filed May 13, 2013.

### BACKGROUND

The invention relates to an endoscope with an endoscope tip, formed at a distal end, at least one imaging optic, arranged in the endoscope tip, and an image recording sensor, arranged in the endoscope tip behind the at least one imaging optic, the at least one imaging optic being held on at least one supporting structure.

Such an endoscope is known for example from DE 10 2007 046 609 A1, in which the image recording sensor is held by a metal tube that is double-plated at least over its inner circumferential surface, a surface layer in the form of a gold layer lying on a nickel layer as a substrate on the metal tube.

It is also known from EP 2 225 998 A1 to provide a protective layer of plastic for the imaging optic with the image recording sensor.

### SUMMARY

The invention is based on the object of providing an endoscope that has a cross-sectional area of the endoscope that is as small as possible.

To achieve this object, it is provided according to the invention that in the endoscope tip there is arranged a metal foil, which is connected to the at least one supporting structure in a tightly sealed manner and which tightly seals off an interior space that receives the at least one image recording sensor. The use according to the invention of a metal foil instead of the previously customary parts produced by machining production methods makes it possible to choose the wall thickness of an encapsulation for the at least one image recording sensor to be as small as possible. In this way, the required cross-sectional area of the endoscope tip can be reduced. The use of a metallic material for the encapsulation of the at least one image recording sensor has the advantage that the encapsulation can be formed so as to be more resistant to thermal loads, as can occur for example in the medical sector during cleaning of the endoscope tip.

In an advantageous embodiment of the invention it may be provided that the endoscope tip has an endoscope tube in which the metal foil is arranged. For example, the endoscope tube may be formed as a metal tube. It is of advantage in this case that the endoscope tube imparts a desired mechanical stability to the endoscope tip. It is also of advantage that between the metal foil and the endoscope tube there can be formed a receiving space for further functions of the endoscope, which is separated from the interior space by the metal foil.

In one embodiment of the invention it may be provided that between the metal foil and an endoscope tube, for example the endoscope tube already mentioned, there is arranged an illuminating optic and/or electronics for the illumination. It is of advantage in this respect that a supply of light to the endoscope tip can be set up. For example, a light source, in particular an LED, may be arranged in the endoscope tip or else in the endoscope, for example at a proximal end of the endoscope. With preference, the illuminating optic is arranged in the receiving space already mentioned and fills it at least partially or even completely in cross section. Instead of the illuminating optic or in addition to it, electronics may also be provided for the preferably direct illumination at the endoscope tip.

In one embodiment of the invention it may be provided that the metal foil has a material thickness of less than 300 μm. More preferably, the metal foil even has a material thickness of less than 50 μm. It is of advantage in this respect that, with the metal foil, wall thicknesses that lie well below the wall thicknesses that can be achieved by machining production methods can be achieved for the encapsulation of the at least one image recording sensor, in particular at acceptable production costs.

Alternatively or in addition, it may be provided that the metal foil has a material thickness of more than 5 μm. With preference, the metal foil has a material thickness of more than 30 μm. It is of advantage in this respect that a tightly sealed, in particular also light-tight, encapsulation of the interior space can be achieved. This is favorable in particular whenever the metal foil is surrounded on the outside by an illuminating optic, in particular the illuminating optic already mentioned, and/or electronics for the illumination. The material thicknesses mentioned offer the advantage here that it is possible to avoid light from the illuminating optic and/or the electronics for the illumination from entering the interior space, and consequently to avoid a disturbance of the image recording of the at least one image recording sensor. The material thicknesses mentioned also have the advantage that the metal foil can be formed as an independent foil body into which the at least one supporting structure and the at least one image recording sensor can be inserted. This allows the production process to be simplified still further.

A range of the material thickness according to the invention that can be used for many applications consequently lies between 5 μm and 300 μm. A range for the material thickness of between 30 μm and 50 μm is particularly favorable.

In one embodiment of the invention it may be provided that the metal foil is produced from wire, inter-wire spaces being closed in a material-bonding manner to form the metal foil. For example, the material-bonding closing may be performed with a soldering material. It is of advantage in this respect that the metal foil can be produced in the desired form in a simple way. To form the metal foil, the wire may preferably be wound onto a shaped body. Alternatively or in addition, the wire may be braided to form the metal foil. It may also be provided that the spaces are closed in a material-bonding manner to form the metal foil. It may be provided in this case that the individual turns of a winding of the wire are not arranged in parallel or that the metal foil is produced from a number of windings made to cross one another. In this way, an interlinked wire that forms the metal foil after closing the inter-wire spaces can be produced.

In one embodiment of the invention it may be provided that the metal foil is formed as a shrink foil. It is advantageous in this respect that a tight seal can be achieved between the metal foil and the at least one supporting structure in a simple way. It is also of advantage in this respect that, after the thermal treatment, the metal foil clings to the at least one supporting structure, whereby the installation space can be further reduced.

In one embodiment of the invention it may be provided that the metal foil is produced in a coating operation. For example, the coating operation may comprise an electroforming, electroplating, sputtering, vapor depositing and/or spraying process. It is advantageous in this respect that particularly thin metal foils can be achieved. For example, the coating operation may be carried out on a shaped body coated with a release layer. It is advantageous in this respect that the metal foil produced can be provided in the desired form for installation into the endoscope tip. It is also advantageous in this respect that the coating operation does not have to be carried out on the sensitive imaging optic and/or the likewise sensitive at least one image recording sensor.

The metal foil may be produced from a metal alloy.

In one embodiment of the invention it may be provided that the metal foil is produced from copper. The use of copper has the additional advantage that it has good heat conducting properties. Consequently, for example, good heat removal from the at least one image recording sensor encapsulated by the metal foil can be achieved.

Alternatively or in addition, it may be provided that the metal foil is produced from high-grade steel. The use of high-grade steel has the advantage that the metal foil can be produced by deep drawing or tube drawing. This allows the effort that is involved in production to be further reduced.

In one embodiment of the invention it may be provided that in the endoscope tip there is arranged a printed circuit board that carries the at least one image recording sensor and/or an electronic circuit connected to the at least one image recording sensor. It is advantageous in this respect that contacting of the at least one image recording sensor and/or electronic pre-processing of output signals of the at least one image recording sensor can be provided in the endoscope tip.

It is particularly favorable in this respect if the printed circuit board is produced from a ceramic material. This has the advantage that the printed circuit board can be formed so as to be resistant to thermal loads that can occur in the medical area of use, for example during cleaning operations. The printed circuit board may also be produced from a plastic.

In one embodiment of the invention it may be provided that the printed circuit board already mentioned has a metallized edge. It is advantageous in this respect that a material-bonding connection to the metal foil can be achieved in a simple manner. The metallized edge is preferably formed peripherally around the printed circuit board, in order to achieve complete sealing-off of the interior space.

In one embodiment of the invention it may be provided that the metal foil is connected in a material-bonding manner to a metallized edge, in particular the metallized edge already mentioned, of a printed circuit board, in particular the printed circuit board already mentioned. It is advantageous in this respect that the interior space taken up by the metal foil can be closed in a tightly sealed manner in a simple way. It is also advantageous in this respect that the metal foil can be held and can be positioned in a simple manner by the printed circuit board. The material-bonding connection may be established for example by adhesive bonding, soldering or welding.

In one embodiment of the invention it may be provided that the metal foil has at least one outlet opening for at least one feed line of the at least one image recording sensor, the outlet opening being closed in a tightly sealed manner. For example, the outlet opening may be filled with a casting compound. Epoxy resin or other casting material can be used for example for this purpose. It is of advantage in this respect that the output signals of the at least one image recording sensor can be led out from the encapsulated interior space.

Simplifications of technical aspects of production are obtained if the metal foil forms a foil body that is inserted in the endoscope tip. It is of advantage in this respect that the metal foil can be produced separately from the sensitive at least one imaging optic and/or the likewise sensitive image recording sensor.

For example, it may be provided that the foil body is formed as a deep-drawn part or tube-drawn part. It is of advantage in this respect that the foil body can be formed in a simple way before the insertion of the at least one imaging optic.

Alternatively or in addition, it may be provided that the foil body is folded and/or bent from a basic body, corresponding connecting edges of the foil body being connected to one another in a material-bonding manner. With preference, the foil body is in this case folded and/or bent from a flat basic body. It is of advantage in this respect that more complex contours and/or forms can be formed.

In one embodiment of the invention it may be provided that the foil body is formed with a non-round cross section. For example, the cross section may be formed as polygonal, in particular oval, triangular, rectangular, pentagonal, hexagonal or with more than six corners, it also being possible for the corners to be respectively rounded. It is of advantage in this respect that the foil body can be inserted into an endoscope tube with a round cross section, so that at least one receiving space for further functions of the endoscope can be formed between the foil body and the endoscope tube.

In one embodiment of the invention it may be provided that the foil body is formed as tubular. The foil body may in this case have a round or non-round cross section. It is of advantage in this respect that the tubular foil body provides an interior space in which the at least one image recording sensor can be arranged. It is also of advantage in this respect that the tubular foil body provides an opening through which the at least one image recording sensor can record images.

In one embodiment of the invention it may be provided that the foil body is closed in a distal region by the at least one imaging optic and/or a glass cover. It is of advantage in this respect that a tight seal of the interior space with respect to the outside can be achieved. The foil body is preferably formed in this case as tubular, for example with a round or a non-round cross section.

Alternatively or in addition, it may be provided in this case that the foil body is closed in a proximal region by a printed circuit board, in particular the printed circuit board already mentioned, and/or by the metal foil. It is of advantage in this respect that the interior space can be closed in a tightly sealed manner by simple measures at the proximal region. The foil body is preferably formed in this case as tubular. The metal foil may in this case have the outlet opening already mentioned, which may be closed with a casting compound or in some other way.

In one embodiment of the invention it may be provided that the at least one supporting structure has a mount for the at least one imaging optic. It is advantageous in this respect that the at least one imaging optic can be held in a simple manner in the at least one supporting structure. It is also advantageous in this case that a tightly sealed connection between the at least one imaging optic and the metal foil can be achieved.

To achieve a tight seal of the interior space with respect to the outside, it may be provided that, for sealing, the at least one supporting structure is connected to the at least one imaging optic in a tightly sealed manner. The sealing may be achieved for example by a material-bonding connection.

It is particularly favorable from technical aspects of production if the at least one supporting structure with the at least one image recording sensor is integrated in an optoelectronic subassembly that can be inserted into the endoscope tip. Preferably, the optoelectronic subassembly can be inserted as a unit into the endoscope tip. It is particularly favorable if the optoelectronic subassembly can be inserted into a foil body, in particular the foil body already mentioned, formed from the metal foil. It is advantageous in this respect that the optoelectronic subassembly can be preassembled and that the metal foil can be produced separately from the sensitive components of the optoelectronic subassembly. To form the optoelectronic subassembly, it may consequently be provided that the at least one image recording sensor is connected to the at least one supporting structure. Preferably, it is provided that the optoelectronic subassembly additionally comprises at least one printed circuit board, in particular the printed circuit board already mentioned.

### BRIEF DESCRIPTION OF THE DRAWINGS

the invention will now be described in more detail on the basis of exemplary embodiments, but is not restricted to these exemplary embodiments. Further exemplary embodiments are provided by combining the features of one or more of the claims with one another and/or with one or more features of the exemplary embodiments.

In the figures:

FIG. 1 shows an endoscope according to the invention in a schematic sectional representation,

FIG. 2 shows a part of the endoscope according to the invention shown in FIG. 1 in a schematic exploded representation,

FIG. 3 shows a foil body of an endoscope according to the invention of wound wire,

FIG. 4 shows a further foil body of an endoscope according to the invention of interlinked wire,

FIG. 5 shows a basic body for the production of a folded foil body for an endoscope according to the invention,

FIG. 6 shows the foil body folded from the basic body as shown in FIG. 5,

FIG. 7 shows a foil body of an endoscope according to the invention produced by deep drawing, and

FIG. 8 shows cross-sectional forms of endoscopes according to the invention.

### DETAILED DESCRIPTION OF THE PREFERRED EMBODIMENTS

The same reference signs are used below in all of the figures to designate components that are functionally and/or structurally identical or similar. The respective explanations therefore respectively apply correspondingly to the other figures.

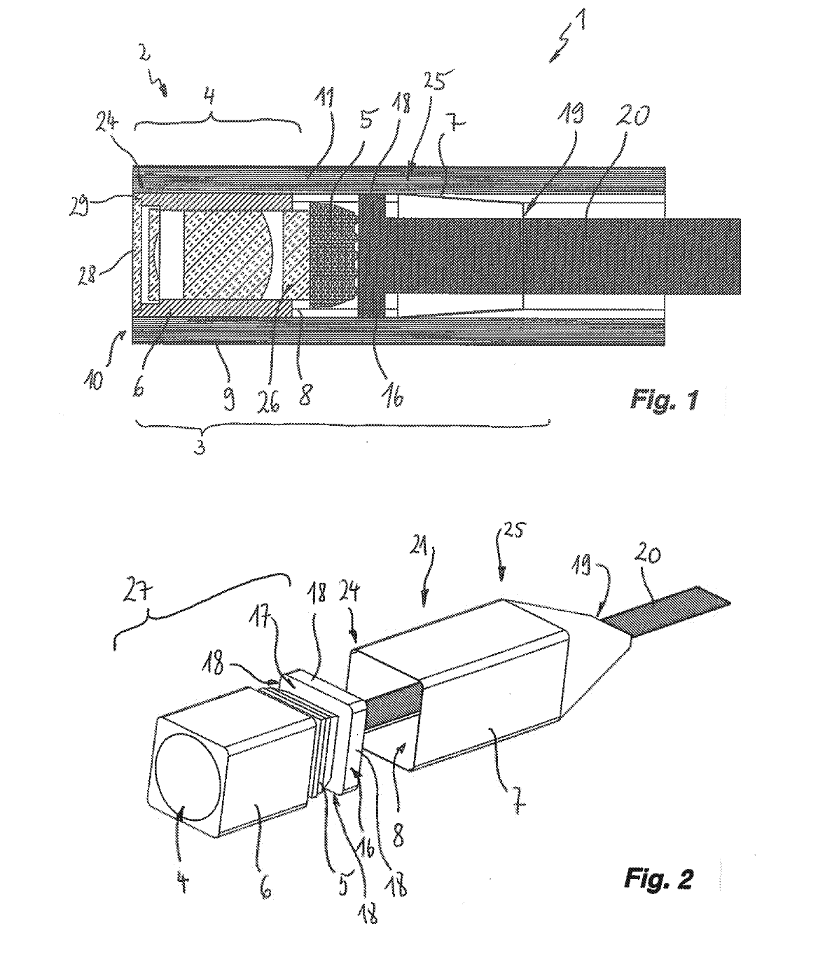

FIG. 1 and FIG. 2 show different views of an endoscope designated as a whole by 1, which are jointly described below.

At the distal end 2 there is formed an endoscope tip 3, in which an imaging optic 4 of the endoscope 1 is arranged.

Arranged behind the imaging optic 4 is an image recording sensor 5.

The imaging optic 4 is arranged and held in a supporting structure 6.

In further exemplary embodiments, the imaging optic 4 may be designed for stereoscopic viewing and comprise a number of imaging optics. These may be arranged on a common supporting structure 6 or on supporting structures that are formed separately from one another. For stereoscopic viewing, two image recording sensors may be used or a common image recording sensor may be formed for both channels.

The supporting structure 6 may in this case be formed as tubular, for example as a round rotary part or a non-round shaped part. The supporting structure 6 is produced from a metallic material.

A metal foil 7 is formed in the endoscope tip 3.

The metal foil 7 is connected to the supporting structure 6 in a tightly sealed manner and encapsulates an interior space 8 in a tightly sealed manner with respect to the outside.

The image recording sensor 5 is arranged in the interior space 8, so that the image recording sensor 5 is encapsulated by the metal foil 7.

On the outside, the endoscope tip 3 has an endoscope tube 9.

The endoscope tube 9 surrounds the metal foil 7 in a radial direction with respect to an axis or running direction of the endoscope 1.

Consequently, a receiving space 10 is formed between the metal foil 7 and the endoscope tube 9.

In further exemplary embodiments, two imaging optics 4 as shown in FIG. 1 may be arranged next to one another, encapsulated in each case with a metal foil 7, and be designed for stereoscopic viewing. These may be arranged on supporting structures that are formed separately from one another. Each imaging optic is then assigned an image recording sensor.

Further functions of the endoscope 1 are arranged in the receiving space 10 in FIG. 1.

For example, FIG. 1 shows that in the receiving space 10 there is arranged an illuminating optic 11, with which a cavity that is being investigated can be illuminated. The illuminating optic 11 may be formed as a fiber optic.

This illuminating optic 11 is connected in a way known per se to a light source that is not represented any further, for example an LED.

In further exemplary embodiments, instead of the illuminating optic 11 or in addition to it, electronics for the illumination, for example with feed lines, LEDs and/or control electronics, are arranged in the receiving space.

Since the schematized sectional representation as shown in FIG. 1 is not to scale, it should be mentioned that, in the exemplary embodiment, the material thickness of the metal foil 7 is selected to be between 30 μm and 50 μm.

In FIG. 2, the endoscope tube 9 and the illuminating optic 11 have been omitted.

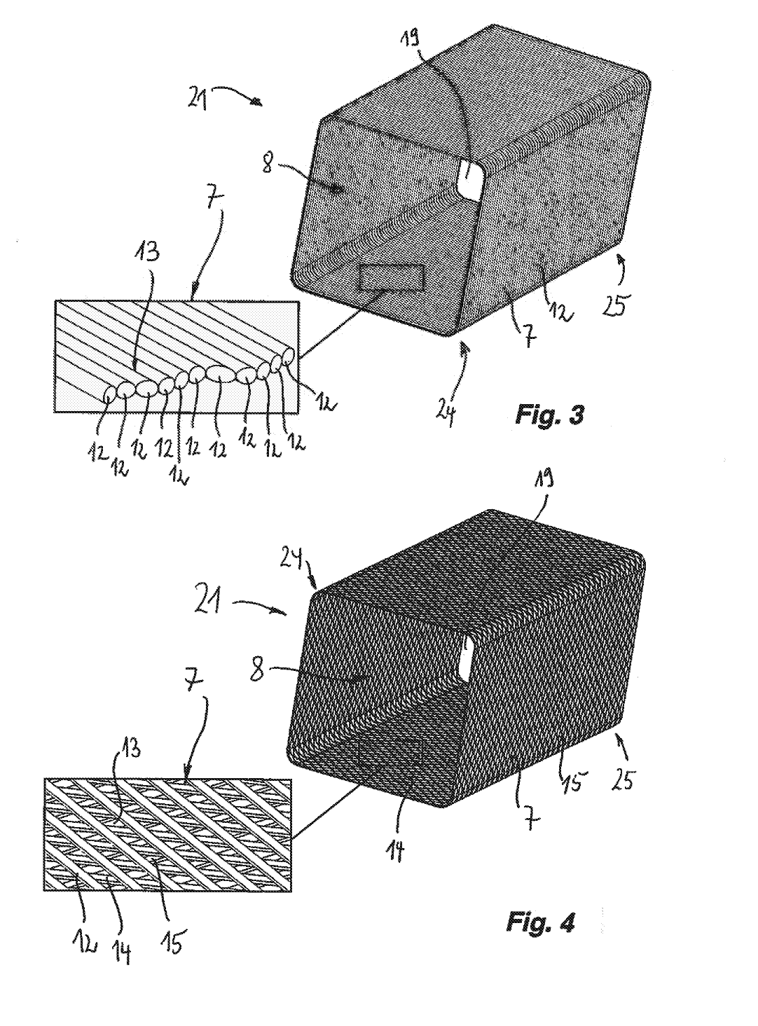

FIG. 3 shows a variant of the metal foil 7 that can be used.

As can be seen from the enlargement of part of the metal foil 7 in FIG. 3, the metal foil 7 is produced from a wound wire 12. This winding has the effect of producing between individual turns of the wire 12 inter-wire spaces 13, which are closed in a material-bonding manner with a soldering material to form the metal foil 7.

In other words, the metal foil 7 does not have a constant material thickness, but instead the material thickness changes between the wires 12 and the inter-wire spaces 13.

FIG. 4 shows a further embodiment of the metal foil 7 that can be used. The metal foil 7 is formed in this case from three windings of wires 12, 14, 15 made to cross one another.

The wires 12, 14, 15 may also be braided with one another, which is not shown any further in FIG. 4 to simplify the representation.

In any case, the wires 12, 14, 15 form between them inter-wire spaces 13, which are closed in a material-bonding manner with a soldering material.

The metal foil 7 as shown in FIG. 4 consequently also does not have a constant material thickness, but a material thickness varying along its extent.

The metal foils 7 as shown in FIG. 3 and FIG. 4 can be used for example in the endoscope 1 as shown in FIG. 1 and FIG. 2.

In a further exemplary embodiment, the metal foil 7 is formed as a shrink foil and is shrink-fitted onto the supporting structure 6.

In further exemplary embodiments, the metal foil 7 is produced in a coating operation such as electroforming, electroplating, sputtering, vapor depositing and/or spraying. After completion of the coating operation, in this case the metal foil is drawn over the supporting structure 6.

The metal foil 7 is produced from copper or from a copper alloy, in order to assist heat dissipation from the image recording sensor 5.

The image recording sensor 5 is arranged and fastened on a ceramic printed circuit board 16.

The printed circuit board 16 additionally carries an electronic circuit 17 known per se, which is not represented any further, for the wiring of the image recording sensor 5 and/or for the processing of output signals of the image recording sensor 5.

The printed circuit board 16 has a peripheral edge 18, which is metallized in a way known per se.

This metallized edge 18 is connected to the metal foil 7 in a material-bonding manner, for example by soldering.

Formed in the metal foil 7 is an outlet opening 19, through which a feed line 20 of the image recording sensor 5 is led.

The outlet opening 19 is filled with an epoxy resin and in this way closed in a tightly sealed manner.

In FIG. 2 it can be seen that the metal foil 7 forms an independent foil body 21.

This foil body 21 can be inserted separately into the endoscope tip 3.

FIG. 7 shows such a foil body 21, which is formed as a deep-drawn part or tube-drawn part.

In a further exemplary embodiment, the foil body 21 is produced in a coating process, the coating being applied to a test body defining the interior space 8. A release layer is applied in this case between the coating and the test body mentioned, so that the metal foil 7 produced can be removed as a foil body 21 from the test body mentioned.

FIG. 5 shows a flat basic body 22 comprising a metal foil 7. The basic body 22 is consequently a blank that can be folded or bent.

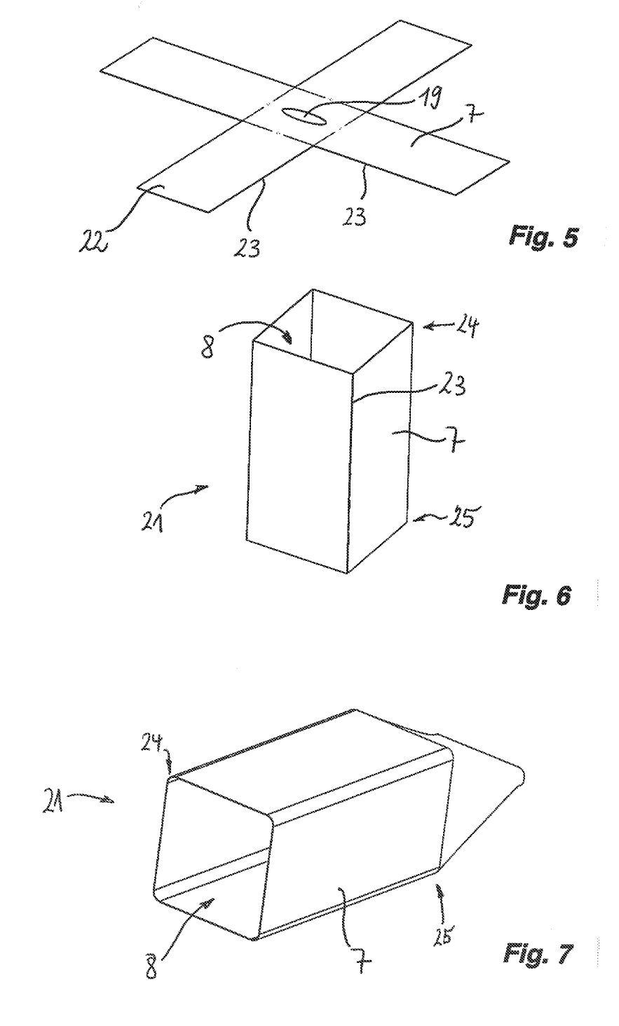

In the folded state, the foil body 21 as shown in FIG. 6 is obtained.

Connecting edges 23 that correspond to one another are in this case connected to one another in a material-bonding manner, for example by soldering.

The connecting edges 23 may in this case also partially overlap, so that a two-dimensional contact is obtained.

In FIGS. 2 to 7, the foil body 21 is depicted with a non-round, rectangular cross section.

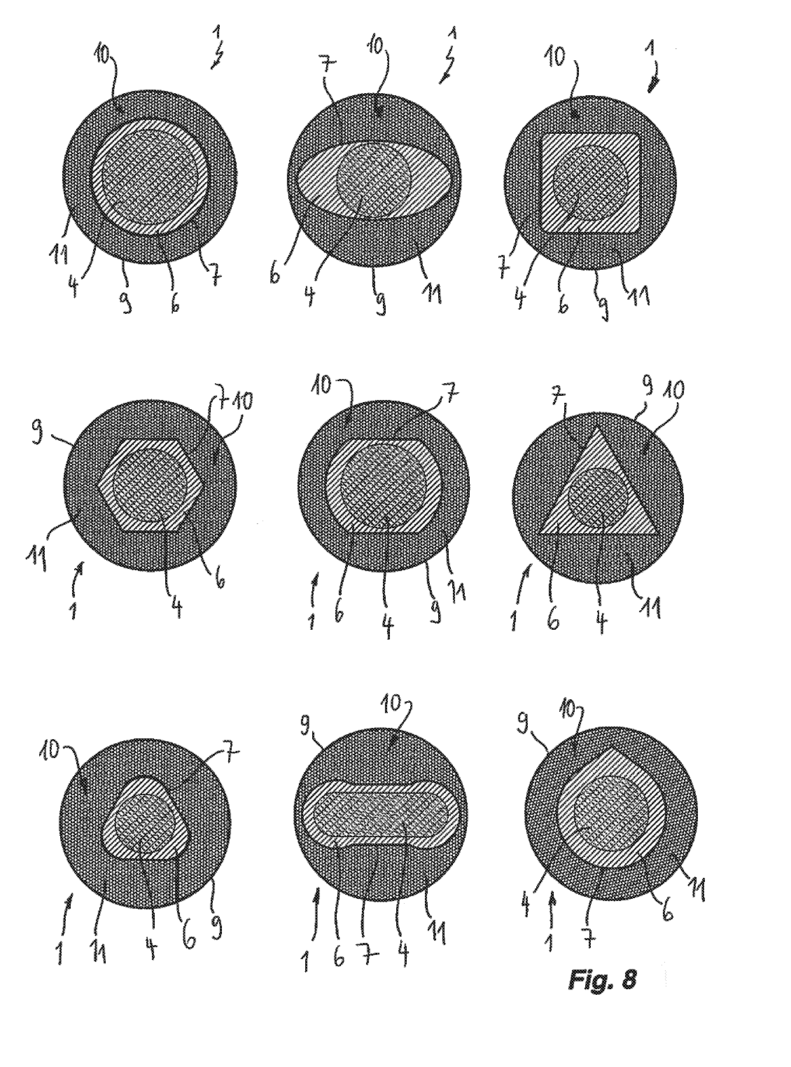

FIG. 8 shows further cross-sectional forms that are realized in further exemplary embodiments.

FIG. 8 specifically shows from left to right and from top to bottom a round cross section, an oval cross section, a rectangular cross section, a hexagonal cross section, a cross section flattened on two sides, a triangular cross section, a triangular-rounded cross section, a spectacle-shaped cross section and a drop-shaped cross section. The cross-sectional forms shown are given by way of example to illustrate that different cross-sectional forms can be used according to the application. Further cross-sectional forms can be used, for example in order to divide the respectively formed receiving space 10 into a number of partial spaces and/or in order to provide specific cross sections of the receiving space 10.

In the exemplary embodiment shown, the foil body 21 is formed as tubular and has a distal region 24 and a proximal region 25.

The distal region 24 is in this case closed by the imaging optic 4, while the proximal region 25 is closed by the printed circuit board 16 and/or the metal foil 7.

In FIG. 1 it can also be seen that the image recording sensor 5 is connected to a glass body 26 of the imaging optic 4.

This results in an optoelectronic subassembly 27, which—as can be seen in FIG. 2—already forms a unit before the installation into the endoscope tip 3.

The foil body 21 can be drawn or fitted over the optoelectronic subassembly 27.

The optoelectronic subassembly 27 can be subsequently inserted with the fitted-on foil body 21 into the endoscope tip 3.

A glass cover 28 is connected to the supporting structure 6 in a material-bonding and tightly-sealing manner in a mount 29. This produces a tight seal of the interior space 8 with respect to the outside.

In the case of an endoscope 1, it is proposed to arrange in an endoscope tip 3 at a distal end 2 of the endoscope 1 an imaging optic 4 and an image receiving sensor 5 in an interior space 8 defined by a metal foil 7.

1. An endoscope (1) comprising an endoscope tip (3), formed at a distal end (2), at least one imaging optic (4) arranged in the endoscope tip (3), and at least one image recording sensor (5) arranged in the endoscope tip (3) behind the at least one imaging optic (4), the at least one imaging optic (4) being held on at least one supporting structure (6), a metal foil (7) arranged in the endoscope tip (3), the metal foil (7) is connected to the at least one supporting structure (6) in a tightly sealed manner that tightly seals off an interior space (8) that receives the at least one image recording sensor (5).

2. The endoscope (1) as claimed in claim 1, wherein the endoscope tip (3) has an endoscope tube (9) in which the metal foil (7) is arranged.

3. The endoscope (1) as claimed in claim 1, wherein between the metal foil (7) and the endoscope tube (9) there is arranged at least one of an illuminating optic (11) or electronics for the illumination.

4. The endoscope (1) as claimed in claim 1, wherein the metal foil (7) has a material thickness of more than 5 μm and less than 300 μm.

5. The endoscope (1) as claimed in claim 1, wherein the metal foil (7) is produced from at least one of a wound, braided, or interlinked wire (12, 14, 15), with inter-wire spaces (13) between the wire being closed in a material-bonding manner.

6. The endoscope (1) as claimed in claim 1, wherein the inter-wire spaces (13) between the wire are closed with a soldering material to form the metal foil (7).

7. The endoscope (1) as claimed in claim 1, wherein the metal foil (7) is formed as a shrink foil.

8. The endoscope (1) as claimed in claim 1, wherein the metal foil (7) is produced in a coating operation by at least one of electroforming, electroplating, sputtering, vapor depositing, or spraying.

9. The endoscope (1) as claimed in claim 1, wherein the metal foil (7) is produced from at least one of copper or from high-grade steel.

10. The endoscope (1) as claimed in claim 1, wherein in the endoscope tip (3) there is arranged a printed circuit board (16) that carries at least one of the image recording sensor (5) or an electronic circuit (17) connected to the at least one image recording sensor (5).

11. The endoscope (1) as claimed in claim 10, wherein the printed circuit board (16) is produced from a ceramic material or from plastic.

12. The endoscope (1) as claimed in claim 10, wherein the printed circuit board (16) has a peripheral metallized edge (18), and the metal foil (7) is connected in a material-bonding manner to the metallized edge (18) of the printed circuit board (16).

13. The endoscope (1) as claimed in claim 1, wherein the metal foil (7) has at least one outlet opening (19) for at least one feed line (20) of the at least one image recording sensor (5), the outlet opening (19) being closed in a tightly sealed manner.

14. The endoscope (1) as claimed in claim 1, wherein the metal foil (7) forms a foil body (21) that is inserted in the endoscope tip (3).

15. The endoscope (1) as claimed in claim 1, wherein the foil body (21) is formed as at least one of a deep-drawn part, a tube-drawn part, or is folded or bent from a basic body (22), with corresponding connecting edges (23) of the foil body (21) being connected to one another in a material-bonding manner.

16. The endoscope (1) as claimed in claim 1, wherein the foil body (21) is formed with a non-round cross section or is formed as tubular.

17. The endoscope (1) as claimed in claim 1, wherein the foil body (21) is tubular and is closed in a distal region (24) by at least one of the at least one imaging optic (4) or a glass cover (28).

18. The endoscope (1) as claimed in claim 10, wherein the foil body (21) is tubular and is closed in a proximal region (25) by at least one of the printed circuit board (16) or by the metal foil (7).

19. The endoscope (1) as claimed in claim 1, wherein the at least one supporting structure (6) has a mount (29) for the at least one imaging optic (4) and, for sealing, the at least one supporting structure (6) is connected to the at least one imaging optic (4) in a tightly sealed manner.

20. The endoscope (1) as claimed in claim 14, wherein the at least one supporting structure (6) with the at least one image recording sensor (5) is integrated in an optoelectronic subassembly (27) that is inserted into the endoscope tip (3) into the foil body (21) formed from the metal foil (7).
